(19) United States
(12) Patent Application Publication
(10) Pub. No.: US 2022/0369734 A1
(43) Pub. Date: Nov. 24, 2022

(54) FOGLESS FACE MASK

(71) Applicant: Brian Kennedy, Ann Arbor, MI (US)

(72) Inventor: Brian Kennedy, Ann Arbor, MI (US)

(21) Appl. No.: 17/479,128

(22) Filed: Sep. 20, 2021

Related U.S. Application Data
(60) Provisional application No. 63/258,722, filed on May 24, 2021.
(60) Provisional application No. 63/258,721, filed on May 24, 2021.

(51) Int. Cl. A41D 13/11

(52) U.S. Cl. CPC A41D 13/1161; A41D 13/1115

(57) ABSTRACT

The present invention includes a mask body having a cord attached at three locations on the mask body and a deformable bridge piece. The cord is attached at the middle of an upper edge of the mask body generally aligning with the location of the bridge of the wearer's nose. The cord is further attached at each of two lower positions on the mask body, preferably near or at the bottom corners of the mask body. The cord is further threaded through eyelets located at each of two upper positions on the mask body, preferably near or at the upper corners of the mask body. The deformable bridge piece is located along the upper edge of the mask body, and generally centered on the bridge of the wearer's nose.

### CLAIM OF PRIORITY

The present application claims the benefit of provisional application 63/258,721, filed May 15, 2021, and provisional application 63/258,722, filed May 15, 2021, both incorporated by reference.

### FIELD OF THE INVENTION

The present invention relates to an improved face mask.

### BACKGROUND OF THE INVENTION

For people who wear glasses, wearing a face mask can be troublesome. Exhaled breath tends to fog glasses. Known solutions include going without glasses, applying anti-fog coating to the lenses, or an improved mask that prevents exhaled breadth from escaping upward on the wearer's face.

One known mask, from U.S. Pat. No. 8,910,634, utilizes apertures through which a cord is threaded and a resilient bridge piece. However, the placement of the apertures along the top edge of mask body, combined with a resilient bridge piece, tends to force the mask body away from the wearer's face thus allowing exhaled breadth to fog the lenses of the wearer's glasses. Other known masks that attempt to be fogless tend to be complex fit properly to the wearer's face and expensive to manufacture.

### SUMMARY OF THE INVENTION

The present invention includes a mask body having a cord attached at three locations on the mask body and a deformable bridge piece. The cord is attached at the middle of an upper edge of the mask body generally aligning with the location of the bridge of the wearer's nose. The cord is further attached at each of two lower positions on the mask body, preferably near or at the bottom corners of the mask body. The cord is further threaded through eyelets located at each of two upper positions on the mask body, preferably near or at the upper corners of the mask body. The deformable bridge piece is located along the upper edge of the mask body, and generally centered on the bridge of the wearer's nose.

### BRIEF DESCRIPTION OF THE DRAWINGS

FIG. 1 shows a front view of one embodiment of the invention.

FIG. 2 shows a top view of one embodiment of the invention.

### DETAILED DESCRIPTION

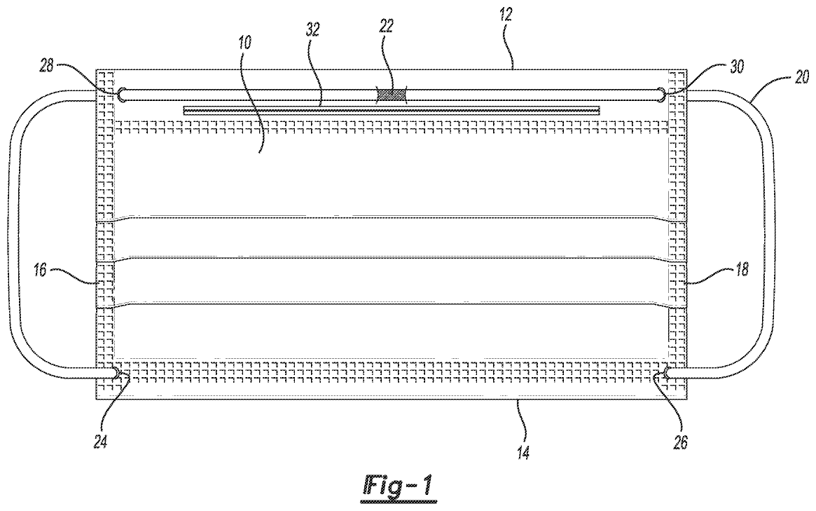

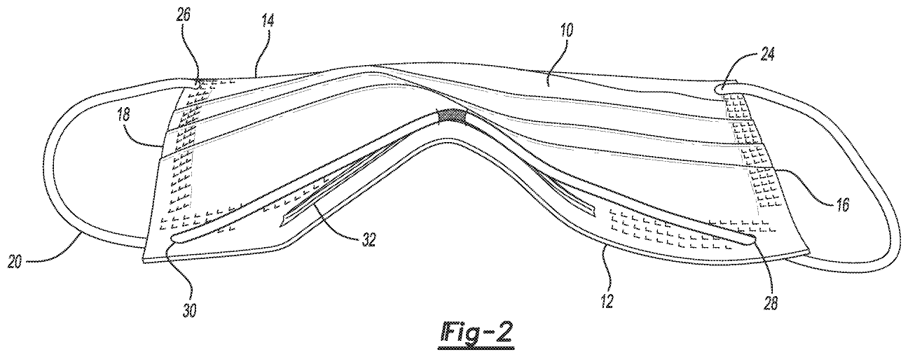

As seen in FIGS. 1 and 2, the invention includes a mask body 10 that is generally has four edges, a top edge 12, a bottom edge 14, and two side edges 16, 18. A cord 20 is attached 22 at or near the top edge of the mask body, preferably in the middle of the edge, aligning with the wearer's nose bridge. The cord is also attached near the bottom corners 24, 26 of the mask body. The cord is threaded through an eyelet near each of the top corners 28, 30 of the mask body. A deformable bridge piece 32 is located near the top edge of the mask body.

When worn, the cord extends around the wearer's ears or head to hold the mask body against the wearer's face and the deformable bridge piece is conformed to the shape of the wearer's nose and cheeks to prevent exhaled breadth from escaping the top edge of the mask.

Air permeable mask bodies are preferred because such mask bodies help trap aerosols in exhaled breadth or otherwise help filter exhaled breadth. While rectilinear mask bodies are preferred, any shape may be used. Furthermore, single layer, multi-layer, and pleated mask bodies may be used. While mask bodies of paper and plastic (such as surgical masks) are preferred, woven and unwoven cloth mask bodies are also contemplated.

The cord is distally attached to the mask body with respect to the deformable bridge piece or the mask body. In one preferred embodiment, the cord is attached to the outside of the mask at the nose bridge; that is, on the side away from the wearer's face, as seen in FIG. 2. When tensioned, the cord attached to the outside tends to force the mask body toward the wearer's face. The cord is further attached to the mask body near the bottom corners, on either the bottom edge or on the side edges. Preferably, the cord is attached at the bottom corners or within about 10% of the length of the edge.

The cord may be attached to the mask body by any method that will withstand the tension applied to the cord while the mask is being donned, doffed, or worn. Suitable attachment methods include adhesive, heat staking, etc.

While the cord preferably is a single length of material, multiple lengths of material may combined into a single length. Even though the cord preferably is held in place by the wearer's ears, cords that are held in place at the back of the wearer's head are also contemplated. The cord may include adjustment devices to insure a comfortable fit on the wearer's ear or head.

The eyelets through which the cord is threaded allow the cord to move relative to the mask body. When tensioned, the cord will slide through the eyelets. The eyelets are located near the top corners, on either the tops edge or on the side edges. Preferably the eyelets are at the top corners and positioned so that there is sufficient remaining material to prevent the cord from tearing or disengaging from the mask body when the cord is tensioned. In one embodiment, the eyelets are positioned within about 10% of the length of the edge. In this manner, the mask body is not held close to the cord, except at the eyelets and the nose bridge of the wearer.

The deformable bridge piece is proximally located relative to the cord; that is, the deformable bridge piece is between the cord and the wearer's face. The deformable bridge piece may be integrated between layers of the mask body or attached to the mask body, either adjacent to the wearer's face or outside of the material of the mask body.

By having the cord on top of it (i.e. distal), the deformable bridge piece better holds the mask body to the wearer's face while the cord applies pressure at the wearer's nose bridge. The absence of eyelets along the top edge of the mask body allows the mask body to be held in place by the deformable bridge piece.

The deformable bridge piece may be made of any material that retains its shape upon being deformed. In one embodiment, the deformable bridge piece is malleable and deformed at the time of donning the mask by the wearer. This provides the closet fit of the mask body to the wearer's face. In another embodiment, the deformable bridge piece is a resilient material with pre-formed 3D shape generally shaped to go over the bridge of the wearer's nose and extend across the wearer's cheeks. A malleable, rather than a resilient, material is preferred with a metal wire being one suitable embodiment.

It will be further appreciated that functions or structures of a plurality of components or steps may be combined into a single component or step, or the functions or structures of one-step or component may be split among plural steps or components. The present invention contemplates all of these combinations. Unless stated otherwise, dimensions and geometries of the various structures depicted herein are not intended to be restrictive of the invention, and other dimensions or geometries are possible. Plural structural components or steps can be provided by a single integrated structure or step. Alternatively, a single integrated structure or step might be divided into separate plural components or steps. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention. The present invention also encompasses intermediate and end products resulting from the practice of the methods herein. The use of “comprising” or “including” also contemplates embodiments that “consist essentially of” or “consist of” the recited feature.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

1. A device comprising:
an air permeable mask body with a top edge, a pair of top corners, and a pair of bottom corners, wherein the mask body includes an eyelet near each top corner,
a deformable bridge piece located at or near the top edge of the mask body such that the deformable bridge piece is attached to or incorporated into the mask body for an entire length of the deformable bridge piece, and
a cord attached near the top edge of the mask body and at the deformable bridge piece, attached near each of the pair of bottom corners, and threaded through each of the eyelets.

2. The device of claim 1 wherein a distance between each top corner and each of the eyelets is less than about 10% of a length of the top edge of the mask body.

3. The device of claim 2 wherein the deformable bridge piece is adapted to be located proximal to the mask body or a wearer of the mask body relative to the cord.

4. The device of claim 3 wherein the deformable bridge piece comprises a malleable material.

5. The device of claim 4 wherein the mask body is rectilinear in shape.

6. The device of claim 5 wherein the mask body comprises a plurality of layers of material.

7. The device of claim 6 wherein the mask body is pleated.

8. The device of claim 7 wherein the cord consists of a single length of material.

9. The device of claim 1 wherein the cord is attached near the middle of the top edge of the mask body.

10. The device of claim 1 wherein, when worn by a wearer, the cord is attached to the top edge of the mask body near the bridge of a nose of the wearer.

11. The device of claim 1 wherein the cord is adapted to be held in place by the ears of a wearer.

12. The device of claim 1 wherein the cord is adapted to be held in place at the back of the head of a wearer.

13. The device of claim 1 wherein the cord comprises multiple lengths of material.

14. The device of claim 13 wherein the cord is adapted to be held in place by the ears of a wearer.

15. The device of claim 13 wherein the cord is adapted to be held in place at the back of the head of a wearer.

16. The device of claim 1 wherein the mask body is adapted to contact a wearer along the entire length of the deformable bridge piece when worn.

17. The device of claim 16 wherein the mask body is adapted to provide an effective seal to the wearer when worn.

18. The device of claim 1 wherein the mask body includes at most two eyelets, with one eyelet near each top corner.

19. The device of claim 17 wherein the deformable bridge piece is non-resilient.
